(19) United States
(12) Patent Application Publication
(10) Pub. No.: US 2011/0306803 A1
(43) Pub. Date: Dec. 15, 2011

(54) SEPARATION OF GLYCOL MONO-TERTIARY-BUTYL ETHER AND GLYCOL DI-TERTIARY-BUTYL ETHER

(75) Inventors: Myeong Seok KIM, Daejeon (KR); Jae Hoon Uhm, Daejeon (KR); Min Sup Park, Daejeon (KR); Jae Wook Kim, Daejeon (KR)

(73) Assignee: DAELIM INDUSTRIAL CO., LTD., Seoul (KR)

(21) Appl. No.: 13/155,517

(22) Filed: Jun. 8, 2011

(30) Foreign Application Priority Data
Jun. 14, 2010 (KR) 10-2010-0056083

(51) Int. Cl. C07C 41/38; C07C 41/42

(52) U.S. Cl. 568/679; 568/678

(57) ABSTRACT

A method of separating glycol mono-tertiary-butyl ether and glycol di-tertiary-butyl ether by an extraction process using hydrophilic and lipophilic extracting agents is disclosed. The separation method includes the step of separating a mixture including glycol mono-tertiary-butyl ether of the following Chemical formula 1 and glycol di-tertiary-butyl ether of the following Chemical formula 2 with a hydrophilic extracting agent and a lipophilic extracting agent,

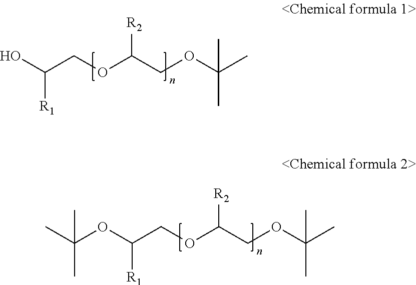

wherein, R₁ and R₂ are independently, a hydrogen atom or an alkyl group of carbon number of 1 to 5, and n is an integer of 0 to 4.

### REFERENCE TO RELATED APPLICATIONS

This application claims the priority benefits of Korean Patent Application No. 10-2010-0056083 filed on Jun. 14, 2010. All disclosures of the Korean Patent application are incorporated herein by reference.

### FIELD OF THE INVENTION

This invention relates to a separation of glycol mono-tertiary-butyl ether and glycol di-tertiary-butyl ether, and more particularly, to a method of separating glycol mono-tertiary-butyl ether and glycol di-tertiary-butyl ether by an extraction process using hydrophilic and lipophilic extracting agents from a mixture of glycol mono-tertiary-butyl ether and glycol di-tertiary-butyl ether.

### BACKGROUND OF THE INVENTION

In order to produce glycol mono-tertiary-butyl ether from glycol by a chemical reaction, a separation and purification process should be carried out to remove glycol di-tertiary-butyl ether (byproduct) from the produced glycol mono-tertiary-butyl ether. However, it is not easy to separate the mixture of glycol mono-tertiary-butyl ether and glycol di-tertiary-butyl ether by conventional separation methods since glycol mono-tertiary-butyl ether and glycol di-tertiary-butyl ether has an azeotropic point. Chinese patent Laid-open No. 1065655A discloses an azeotropic distillation of glycol di-tertiary-butyl ether with water at about 98° C., but the azeotropic distillation process requires large amount of water, and large utility cost and energy consumption are inevitable to distillate water and glycol di-tertiary-butyl ether. U.S. Pat. No. 5,552,024 discloses an extraction distillation process using a glycol extracting agent such as dipropyleneglycol to purify dipropyleneglycol tert-butyl ether from impurities. U.S. Pat. Nos. 4,345,102 and 6,730,815 disclose methods to synthesize glycol alkylether compound.

### SUMMARY OF THE INVENTION

Therefore, it is an object of the present invention to provide a separation method of glycol mono-tertiary-butyl ether and glycol di-tertiary-butyl ether which is simple and economical. It is other object of the present invention to provide a separation method of glycol mono-tertiary-butyl ether and glycol di-tertiary-butyl ether in which extracting agents can be recycled.

In order to achieve these and other objects, the present invention provides a separation method of glycol mono-tertiary-butyl ether and glycol di-tertiary-butyl ether, comprising the step of separating a mixture including glycol mono-tertiary-butyl ether of the following Chemical formula 1 and glycol di-tertiary-butyl ether of the following Chemical formula 2 with a hydrophilic extracting agent and a lipophilic extracting agent.

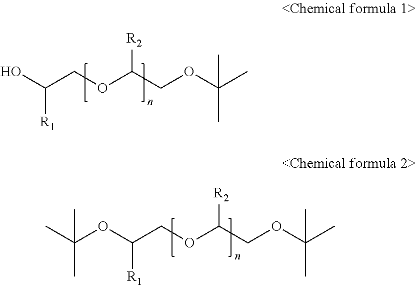

In Chemical formula 1 and 2, R₁ and R₂ are independently, a hydrogen atom or an alkyl group of carbon number of 1 to 5, and n is an integer of 0 to 4.

The method of the present invention can easily separate glycol mono-tertiary-butyl ether and glycol di-tertiary-butyl ether with a relatively simple process (extraction and distillation) by using a hydrophilic extracting agent and a lipophilic extracting agent. The hydrophilic and lipophilic extracting agents can be recovered and recycled after the separation process. The separation method of the present invention requires less amount of energy, and is economically preferable.

### BRIEF DESCRIPTION OF THE DRAWINGS

FIG. 1 is a drawing for illustrating the separation method of glycol mono-tertiary-butyl ether and glycol di-tertiary-butyl ether according to an embodiment of the present invention.

### DETAILED DESCRIPTION OF THE INVENTION

A more complete appreciation of the invention, and many of the attendant advantages thereof, will be better appreciated by reference to the following detailed description.

The separation method of the present invention comprises the step of separating a mixture including glycol mono-tertiary-butyl ether of the following Chemical formula 1 and glycol di-tertiary-butyl ether of the following Chemical formula 2 with a hydrophilic extracting agent and a lipophilic extracting agent. Preferably, the separation method of the present invention comprises the steps of: introducing the mixture including glycol mono-tertiary-butyl ether of the following Chemical formula 1 and glycol di-tertiary-butyl ether of the following Chemical formula 2 into an extraction column, preferably, a multistage extraction column; introducing the lipophilic extracting agent into the bottom (or lower part) of the extraction column and introducing the hydrophilic extracting agent into the top (or upper part) of the extraction column, and operating the extraction column, for example, at the temperature of 10 to 60° C. and at the pressure of atmospheric pressure (1 atm) to 15 bar; and obtaining hydrophilic components (hydrophilic compound layer) including glycol mono-tertiary-butyl ether and the hydrophilic extracting agent from the bottom of the extraction column, and obtaining lipophilic components (lipophilic compound layer) including glycol di-tertiary-butyl ether and the lipophilic extracting agent from the top of the extraction column.

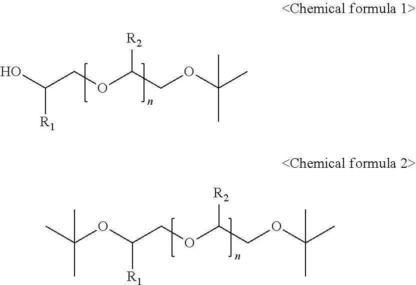

In Chemical formula 1 and 2, R₁ and R₂ are independently, a hydrogen atom or an alkyl group of carbon number of 1 to 5, and n is an integer of 0 to 4 (i.e., 0, 1, 2, 3 or 4).

The mixture used in the present invention includes glycol mono-tertiary-butyl ether and glycol di-tertiary-butyl ether which cannot be easily separated from the mixture by conventional methods. The mixture can be a reaction product of glycol compound of the following Chemical formula 3 and hydrocarbon compound of carbon number 4 (C4 hydrocarbon compound) including isobutene as a reactant which are reacted in the presence of acidic catalyst. Example of the C4 hydrocarbon compound includes a mixture of isobutene and one or more compounds selected from the group consisting of isobutane, n-butane, 1-butene, trans-2-butene, cis-2-butene, 1,3-butadiene, 1,2-butadiene, and so on. Example of the acidic catalyst includes strong acid cation exchange resin, preferably, which has sulfonic acid group (SO₃H) as a strong acid group and is water-insoluble. More specific examples of the acidic catalyst include styrene-sulfonic acid type cation exchange resin, phenol-sulfonic acid type cation exchange resin, cross-linking product thereof, sulfonated coal, sulfonated asphalt, and so on.

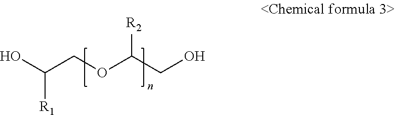

In Chemical formula 3, R₁ and R₂ are independently, a hydrogen atom or an alkyl group of carbon number of 1 to 5, and n is an integer of 0 to 4.

For example, the glycol compound of Chemical formula 3 can be ethylene glycol when n is 0, and R₁ is a hydrogen atom (H), propylene glycol when n is 0, and R₁ is an alkyl group of carbon number 1 (methyl group), dipropylene glycol when n is 1, and R₁ and R₂ are an alkyl group of carbon number 1, triethylene glycol when n is 2, and R₁ and R₂ are a hydrogen atom (H), and tripropylene glycol when n is 2, and R₁ and R₂ are an alkyl group of carbon number 1. The glycol compound can be converted into the (reaction) mixture including glycol mono-tertiary-butyl ether and glycol di-tertiary-butyl ether by the above-mentioned reaction. The reaction product (reaction mixture) of the above-mentioned reaction may include not only glycol mono-tertiary-butyl ether and glycol di-tertiary-butyl ether, but also unreacted glycol compound of Chemical formula 3 and C4 hydrocarbon residue (unreacted C4 hydrocarbon compounds), and so on.

The lipophilic extracting agent used in the present invention extracts lipophilic compound such as glycol di-tertiary-butyl ether from the mixture, and forms a lipophilic compound layer (organic layer). Conventional lipophilic extracting agent can be used as the lipophilic extracting agent of the present invention. The lipophilic extracting agent can be selected from the group consisting of hydrocarbon compounds of carbon number of 2 to 16, preferably 2 to 10, and more preferably 3 to 8, and mixtures thereof. The hydrocarbon compound of carbon number 4 (C4 hydrocarbon compound), which already exists in the reaction mixture, can be preferably used as the lipophilic extracting agent. In this case, the recovery and/or recycling of the lipophilic extracting agent can be easily carried out. Specific examples of the lipophilic extracting agent include ethylene, propylene, n-butane, isobutane, isobutene, 1-butene, 2-butene, 1,2-butadiene, 1,3-butadiene, pentane such as n-pentane, pentene such as 1-pentene, hexane such as n-hexane, hexene such as 1-hexene, mixtures thereof, and so on. One or more of the hydrocarbon compounds can be used as the lipophilic extracting agent. When the carbon number of the lipophilic extracting agent is less than 2, the extraction of glycol di-tertiary-butyl ether cannot be properly carried out. When the carbon number of the lipophilic extracting agent is more than 16, the viscosity of the lipophilic extracting agent increases and the extraction efficiency is deteriorated, and it may be difficult to recover and/or recycle the lipophilic extracting agent due to its increased boiling point. The amount of the lipophilic extracting agent can be varied according to the amount of the lipophilic compound (e.g., glycol di-tertiary-butyl ether) in the reaction mixture.

The hydrophilic extracting agent used in the present invention extracts hydrophilic compound such as glycol mono-tertiary-butyl ether from the mixture, and forms a hydrophilic compound layer (water layer). Conventional hydrophilic extracting agent can be used as the hydrophilic extracting agent of the present invention. The hydrophilic extracting agent can be selected from the group consisting of water, alcohol compounds of carbon number of 1 to 8, preferably 2 to 6, glycol compounds of carbon number of 1 to 10, preferably 2 to 8, and mixtures thereof. Examples of the alcohol compound of carbon number of 1 to 8 include methanol, ethanol, propanol, pentanol, hexanol and so on, and examples of the glycol compound of carbon number 2 to 10 include ethylene glycol, propylene glycol, diethylene glycol, dipropylene glycol, and so on. The amount of the hydrophilic extracting agent can be varied according to the amount of the hydrophilic compound (e.g., glycol mono-tertiary-butyl ether) in the reaction mixture.

The extraction column used in the present invention can be a conventional multistage (for example, 2 to 30 stages) extraction column, and, for example, a countercurrent multistage liquid/liquid extraction column. The operation conditions of the extraction column can be changed according to the pressure of the reaction mixture. Generally, the extraction column can be operated at the temperature of 10 to 60° C., preferably 20 to 40° C. and at the pressure of atmospheric pressure (1 atm) to 15 bar; preferably atmospheric pressure to 12 bar; and more preferably 2 to 10 bar. When the operation temperature of the extraction column is less than 10° C., extra energy is required to operate refrigerant. When the operation temperature of the extraction column is more than 60° C., extra energy is also required to operate a heater. When the operation pressure of the extraction column is less than the atmospheric pressure, extra equipment is required to reduce the pressure. When the operation pressure of the extraction column is more than 15 bar, the extraction column needs to be made of expensive material enduring the high pressure.

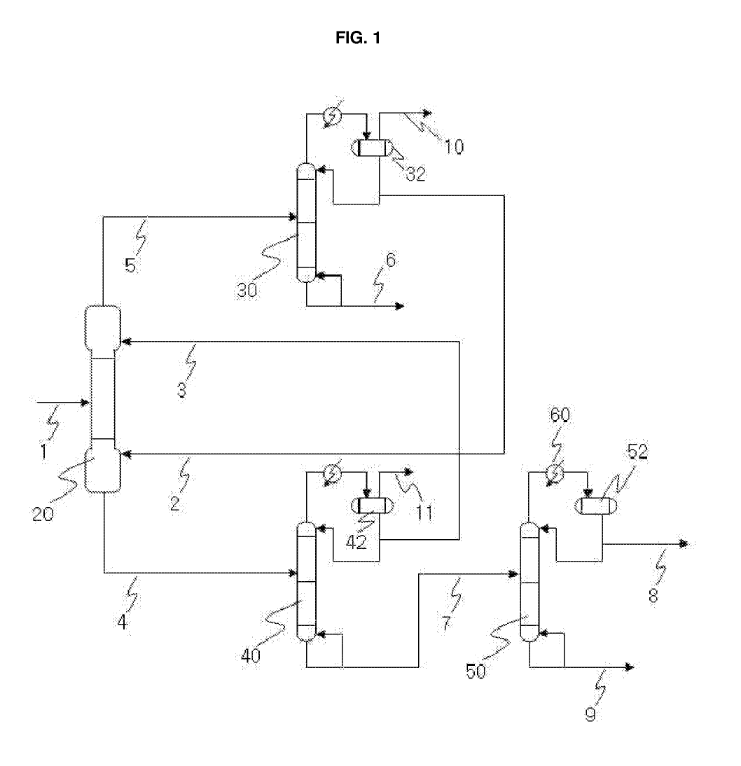

FIG. 1 is a drawing (schematic operation diagram) for illustrating the separation method of glycol mono-tertiary-butyl ether and glycol di-tertiary-butyl ether according to an embodiment of the present invention. In the separation method shown in FIG. 1, a hydrocarbon compound, which is same with the C4 hydrocarbon compound (reaction residue) in the reaction mixture 1, is used as the lipophilic extracting agent 2, and, water, an alcohol compound of carbon number of 1 to 8, and mixtures thereof are used as the hydrophilic extracting agent 3.

As shown in FIG. 1, the reaction mixture 1 including glycol di-tertiary-butyl ether 6, glycol mono-tertiary-butyl ether 8, unreacted glycol compound 9 of Chemical formula 3 and C4 (carbon number of 4) hydrocarbon compound 10 (reaction residue) is introduced into an extraction column 20. Then, a lipophilic extracting agent 2 is introduced into the bottom (or lower part) of the extraction column 20, and a hydrophilic extracting agent 3 is introduced into the top (or upper part) of the extraction column 20. The lipophilic extracting agent 2 and lipophilic compounds such as glycol di-tertiary-butyl ether 6 form a lipophilic compound layer 5 (organic layer), and move from the lower part to the upper part of the extraction column 20. The hydrophilic extracting agent 3 and hydrophilic compounds such as glycol mono-tertiary-butyl ether 8 form a hydrophilic compound layer 4 (water layer), and move from the upper part to the lower part of the extraction column 20. The boundary between the organic layer 5 and the water layer 4 can be formed on the upper part or on the lower part of the extraction column 20 according to the amounts of the extracting agents, operation conditions, and so on. When the boundary is formed on the upper part of the extraction column 20, the water layer 4 is the dominant layer and the lipophilic extracting agent 2 extracts the lipophilic compounds to the top of the extraction column 20. When the boundary is formed on the lower part of the extraction column 20, the organic layer 5 is the dominant layer and the hydrophilic extracting agent 3 extracts the hydrophilic compounds to the bottom of the extraction column 20. By operating the extraction column 20, the hydrophilic compound layer 4 (water layer) is obtained from the bottom of the extraction column 20, and the lipophilic compound layer 5 (organic layer) is obtained from the top of the extraction column 20. Thereby, glycol mono-tertiary-butyl ether 8 and glycol di-tertiary-butyl ether 6 are separated in the forms of the hydrophilic compound layer 4 and the lipophilic compound layer 5.

The hydrophilic compound layer 4 (water layer) obtained from the bottom of the extraction column 20 is distilled at the first distillation column 40 to separate glycol mono-tertiary-butyl ether 8 and the hydrophilic extracting agent 3, and the separated hydrophilic extracting agent 3 is reintroduced into the top (or upper part) of the extraction column 20. The lipophilic compound layer 5 (organic layer) obtained from the top of the extraction column 20 is distilled at the second distillation column 30 to separate glycol di-tertiary-butyl ether 6 and the lipophilic extracting agent 2, and the separated lipophilic extracting agent 2 is reintroduced into the bottom (or lower part) of the extraction column 20. Thereby, the hydrophilic compound layer 4 can be separated into glycol mono-tertiary-butyl ether 8 and the hydrophilic extracting agent 3, and the lipophilic compound layer 5 can be separated into glycol di-tertiary-butyl ether 6 and the lipophilic extracting agent 2.

The first distillation column 40 can be a conventional distillation column, more specifically, a conventional multistage distillation column, and, for example, can be a distillation column having 5 to 50 stages. The hydrophilic extracting agent 3 is recovered from the top of the first distillation column 40, and reintroduced into the extraction column 20 through the first storage tank 42. The other hydrophilic reactants except the hydrophilic extracting agent 3, namely, glycol mono-tertiary-butyl ether 8 and unreacted glycol compound 9 of Chemical formula 3 are discharged from the bottom of the first distillation column 40. The temperature of the top of the first distillation column 40 can be a boiling point of the hydrophilic extracting agent 3 at the operating pressure of the first distillation column 40. For example, when water is used as the hydrophilic extracting agent 3, and the operating pressure of the first distillation column 40 is 0.1 bar, the temperature of the top of the first distillation column 40 is about 45° C. When water is used as the hydrophilic extracting agent 3, and the operating pressure of the first distillation column 40 is atmospheric pressure, the temperature of the top of the first distillation column 40 is about 100° C. The temperature of the bottom of the first distillation column 40 can be a boiling point of a mixture 7 of glycol mono-tertiary-butyl ether 8 and unreacted glycol compound 9 at the operating pressure of the first distillation column 40.

The separation method of the present invention may further include the step of purifying glycol mono-tertiary-butyl ether 8 and separating (removing) the unreacted glycol compound 9 by distilling the mixture 7 of glycol mono-tertiary-butyl ether 8 and unreacted glycol compound 9 at the third distillation column 50. The third distillation column 50 can be a conventional distillation column, more specifically, a conventional multistage distillation column, and, for example, can be a distillation column having 5 to 50 stages. The glycol mono-tertiary-butyl ether 8 is recovered from the top of the third distillation column 50, a heat exchanger 60 and the third storage tank 52. The unreacted glycol compound 9 is discharged from the bottom of the third distillation column 50. The operation pressure of the third distillation column 50 can be 0.05 to 0.5 bar, preferably 0.08 to 0.2 bar. The temperature of the top of the third distillation column 50 can be a boiling point of glycol mono-tertiary-butyl ether 8 at the operating pressure of the third distillation column 50. The temperature of the bottom of the third distillation column 50 can be a boiling point of the unreacted glycol compound 9 at the operating pressure of the third distillation column 50. When the operation pressure of the third distillation column 50 is less than 0.05 bar, extra equipment and cost are required to reduce the pressure. When the operation pressure of the third distillation column 50 is more than 0.5 bar, extra energy is required to operate the third distillation column 50 and it is economically unfavorable.

The second distillation column 30 can be a conventional distillation column, more specifically, a conventional multistage distillation column, and, for example, can be a distillation column having 5 to 50 stages. The lipophilic extracting agent 2 is recovered from the top of the second distillation column 30, and reintroduced into the extraction column 20 through the second storage tank 32. In FIG. 1, a stream of C4 hydrocarbon compound 10 includes excess lipophilic extracting agent 2 and C4 hydrocarbon compound (C4 hydrocarbon residue, unreacted C4 hydrocarbon compounds) which is reaction residue in the reaction mixture 1, and the stream can be recovered and kept for other uses. The other reactant except the stream of C4 hydrocarbon compound 10, namely, glycol di-tertiary-butyl ether 6 is discharged from the bottom of the second distillation column 30. The operation pressure of the second distillation column 30 can be controlled according to the kind of the lipophilic extracting agent 2. For example, when the lipophilic extracting agent 2 of carbon number of equal or less than 4 is used, the operation pressure of the second distillation column 30 is about 4 bar. When the lipophilic extracting agent 2 of carbon number of 5 to 8 is used, the operation pressure of the second distillation column 30 is atmospheric pressure (1 atm). When the lipophilic extracting agent 2 of carbon number of equal or more than 9 is used, the operation pressure of the second distillation column 30 is a reduced pressure. The temperature of the top of the second distillation column 30 can be a boiling point of lipophilic extracting agent 2 at the operating pressure of the second distillation column 30. The temperature of the bottom of the second distillation column 30 can be a boiling point of glycol di-tertiary-butyl ether 6 at the operating pressure of the second distillation column 30 (for example, atmospheric pressure to 2 bar).

Hereinafter, the preferable examples are provided for better understanding of the present invention. However, the present invention is not limited by the following examples.

### Example 1

### Separation of Diethyleneglycol Mono-Tertiary-Butyl Ether and Diethyleneglycol Di-Tertiary-Butyl Ether

A reaction mixture 1 including diethyleneglycol mono-tertiary-butyl ether (DETB) 84.99 weight % and diethyleneglycol di-tertiary-butyl ether (DDBE) 15.01 weight % shown in Table 1 (Stream number 1) is separated with an extraction column 20, and the first, the second, and the third distillation columns 30, 40, 50 shown in FIG. 1 according to the present invention. The material flows (unit: kg/hr) in the reaction mixture 1, lipophilic extracting agent 2, hydrophilic extracting agent 3, hydrophilic compound layer 4, lipophilic compound layer 5, glycol di-tertiary-butyl ether 6, mixture 7 of glycol mono-tertiary-butyl ether 8 and unreacted glycol compound 9, glycol mono-tertiary-butyl ether 8, unreacted glycol compound 9 and C4 hydrocarbon compound 10, temperature, and pressure are also shown in Table 1.

TABLE 1

| | Stream number | | | | | | | | | |
|---|---|---|---|---|---|---|---|---|---|---|
| | 1 | 2 | 3 | 4 | 5 | 6 | 7 | 8 | 9 | 10 |
| Propane | 0.4 | 0.2 | 0.0 | 0.1 | 0.5 | 0.0 | 0.0 | 0.0 | 0.0 | 0.4 |
| Isobutane | 34.4 | 13.4 | 0.0 | 3.3 | 44.5 | 0.0 | 0.0 | 0.0 | 0.0 | 31.1 |
| n-butane | 141.9 | 57.0 | 0.1 | 6.6 | 192.4 | 3.0 | 0.0 | 0.0 | 0.0 | 132.4 |
| 1-butene | 390.5 | 156.2 | 0.5 | 26.5 | 520.7 | 1.7 | 0.0 | 0.0 | 0.0 | 362.8 |
| Isobutene | 71.6 | 25.6 | 0.2 | 12.1 | 85.4 | 0.2 | 0.0 | 0.0 | 0.0 | 59.6 |
| trans-2-butene | 137.3 | 48.9 | 0.5 | 21.0 | 165.7 | 3.1 | 0.0 | 0.0 | 0.0 | 113.7 |
| cis-2-butene | 71.4 | 24.8 | 0.3 | 10.7 | 85.8 | 3.4 | 0.0 | 0.0 | 0.0 | 57.6 |
| 1,3-butadiene | 0.0 | 0.0 | 0.0 | 0.0 | 0.0 | 0.0 | 0.0 | 0.0 | 0.0 | 0.0 |
| 1,2-butadiene | 7.2 | 1.8 | 0.1 | 1.2 | 7.8 | 1.9 | 0.0 | 0.0 | 0.0 | 4.1 |
| Water (H₂O) | 0.3 | 0.8 | 932.7 | 931.2 | 2.5 | 0.0 | 0.3 | 0.3 | 0.0 | 1.8 |
| DETB | 1255.5 | 0.0 | 69.4 | 1122.8 | 102.0 | 102.0 | 1053.3 | 1149.9 | 3.4 | 0.0 |
| DDBE | 221.7 | 0.0 | 0.0 | 0.0 | 221.7 | 221.7 | 0.0 | 0.0 | 0.0 | 0.0 |
| DEG | 727.8 | 0.0 | 0.0 | 727.8 | 0.0 | 0.0 | 727.8 | 10.3 | 717.5 | 0.0 |
| Total | 3060.0 | 328.6 | 1003.8 | 2863.3 | 1429.1 | 337.1 | 1781.4 | 1160.5 | 720.9 | 763.4 |
| Temp (° C.) | 35.0 | 44.9 | 45.2 | 37.6 | 42.9 | 203.5 | 231.2 | 45.0 | 178.4 | 44.9 |
| Pressure | 10.0 | 10.0 | 10.0 | 10.0 | 10.0 | 4.2 | 0.4 | −0.97 | −0.90 | 10.0 |
| (kgf/cm² · G) | | | | | | | | | | |

DETB: diethyleneglycol mono-tertiary-butyl ether,
DDBE: diethyleneglycol di-tertiary-butyl ether,
DEG: diethyleneglycol

### Example 2

### Separation of Diethyleneglycol Mono-Tertiary-Butyl Ether and Diethyleneglycol Di-Tertiary-Butyl Ether

A reaction mixture 1 including diethyleneglycol mono-tertiary-butyl ether (DETB) 67 weight % and diethyleneglycol di-tertiary-butyl ether (DDBE) 33 weight % is used. Hexane is used as the lipophilic extracting agent 2, and water is used as the hydrophilic extracting agent 3. The flow amount of hexane is controlled to be same with the flow amount of diethyleneglycol di-tertiary-butyl ether (DDBE) in the reaction mixture 1, and the flow amount of water is controlled to be same with the flow amount of diethyleneglycol mono-tertiary-butyl ether (DETB) in the reaction mixture 1. Except the above mentioned conditions, diethyleneglycol mono-tertiary-butyl ether (DETB) and diethyleneglycol di-tertiary-butyl ether (DDBE) are separated according to the method of Example 1. The concentration of diethyleneglycol mono-tertiary-butyl ether (DETB) in the hydrophilic compound layer 4 obtained from the bottom of the extraction column 20 is 99.88 weight %, and the concentration of diethyleneglycol di-tertiary-butyl ether (DDBE) obtained from the top of the extraction column 20 and the distillation column 30 is 98.39 weight %.

From Examples 1 and 2, it is confirmed that glycol mono-tertiary-butyl ether 8 and glycol di-tertiary-butyl ether 6 can be easily separated by using lipophilic and hydrophilic extracting agents 2, 3 and with relatively simple processes (extraction and distillation) according to the present invention. The lipophilic and hydrophilic extracting agents 2, 3 can be recovered and/or recycled after the separation. Therefore, the overall separation process can be operated with less energy and cost.

1. Separation method of glycol mono-tertiary-butyl ether and glycol di-tertiary-butyl ether, comprising the step of:
separating a mixture including glycol mono-tertiary-butyl ether of the following Chemical formula 1 and glycol di-tertiary-butyl ether of the following Chemical formula 2 with a hydrophilic extracting agent and a lipophilic extracting agent, 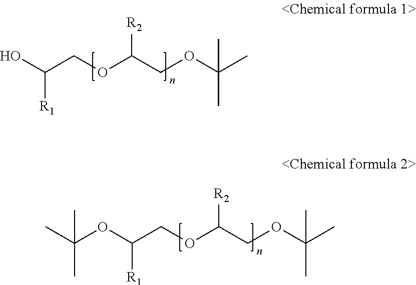
wherein, R₁ and R₂ are independently, a hydrogen atom or an alkyl group of carbon number of 1 to 5, and n is an integer of 0 to 4.

2. The separation method according to claim 1, wherein the method comprises the steps of:
introducing the mixture including glycol mono-tertiary-butyl ether of Chemical formula 1 and glycol di-tertiary-butyl ether of Chemical formula 2 into an extraction column;
introducing the lipophilic extracting agent into a lower part of the extraction column and introducing the hydrophilic extracting agent into an upper part of the extraction column, and operating the extraction column; and
obtaining hydrophilic components including glycol mono-tertiary-butyl ether and the hydrophilic extracting agent from the bottom of the extraction column, and obtaining lipophilic components including glycol di-tertiary-butyl ether and the lipophilic extracting agent from the top of the extraction column.

3. The separation method according to claim 1, wherein the lipophilic extracting agent is selected from the group consisting of hydrocarbon compounds of carbon number of 2 to 16 and mixtures thereof, and the hydrophilic extracting agent is selected from the group consisting of water, alcohol compounds of carbon number of 1 to 8, glycol compounds of carbon number of 1 to 10, and mixtures thereof.

4. The separation method according to claim 2, wherein the extraction column is operated at the temperature of 10 to 60° C. and at the pressure of atmospheric pressure (1 atm) to 15 bar.

5. The separation method according to claim 2, further comprising the steps of:
distilling the hydrophilic components obtained from the bottom of the extraction column with a first distillation column to separate glycol mono-tertiary-butyl ether and the hydrophilic extracting agent, and reintroducing the separated hydrophilic extracting agent into the upper part of the extraction column; and
distilling the lipophilic components obtained from the top of the extraction column with a second distillation column to separate glycol di-tertiary-butyl ether and the lipophilic extracting agent, and reintroducing the separated lipophilic extracting agent into the lower part of the extraction column.

6. The separation method according to claim 5, further comprising the step of purifying glycol mono-tertiary-butyl ether by distilling the glycol mono-tertiary-butyl ether separated from the hydrophilic components with a third distillation column.

7. The separation method according to claim 1, wherein the mixture including glycol mono-tertiary-butyl ether and glycol di-tertiary-butyl ether is a reaction product of glycol compound of the following Chemical formula 3 and hydrocarbon compound of carbon number 4 including isobutene as a reactant which are reacted in the presence of acidic catalyst, 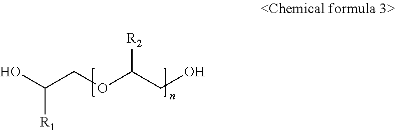
wherein, R₁ and R₂ are independently, a hydrogen atom or an alkyl group of carbon number of 1 to 5, and n is an integer of 0 to 4.
